(19) United States
(12) Patent Application Publication
(10) Pub. No.: US 2021/0140886 A1
(43) Pub. Date: May 13, 2021

(54) FRESHNESS MEASUREMENT SYSTEM

(71) Applicant: TOSHIBA TEC KABUSHIKI KAISHA, Tokyo (JP)

(72) Inventors: Kazuki Taira, Ota Tokyo (JP); Ryozo Akiyama, Mishima Shizuoka (JP); Yukitami Mizuno, Ota Tokyo (JP)

(21) Appl. No.: 16/917,992

(22) Filed: Jul. 1, 2020

(30) Foreign Application Priority Data
Nov. 12, 2019 (JP) 2019-205059

(51) Int. Cl. G01N 21/64; G01N 33/02; G01N 21/76

(52) U.S. Cl. CPC G01N 21/6428; G01N 2021/6439; G01N 21/76; G01N 33/02

(57) ABSTRACT

According to one embodiment, a freshness measurement system capable of quantitatively evaluating the freshness of a perishable food by a simpler method than a related art is provided. A freshness measurement system according to an embodiment includes an irradiation unit, a measurement unit, and a processing unit. The irradiation unit irradiates a phosphor that changes the intensity of fluorescence according to the concentration of a component released from a test subject with an excitation light. The measurement unit measures the intensity of fluorescence emitted by the phosphor. The processing unit determines the freshness index of the test subject using the intensity. The freshness index is, for example, a K value.

### CROSS-REFERENCE TO RELATED APPLICATION

This application is based upon and claims the benefit of priority from Japanese Patent Application No. 2019-173498, filed in Sep. 24, 2019, the entire contents of which are incorporated herein by reference.

### FIELD

Embodiments described herein relate generally to a freshness measurement system and a freshness measurement method.

### BACKGROUND

As a method for evaluating the freshness of a perishable food such as meat or fish, for example, there are various types such as a sensory evaluation method, a chemical evaluation method, and a physical evaluation method. The sensory evaluation method is a method for performing evaluation using a human sense by relying on the color of appearance, damage, smell, sense of touch, or the like of a perishable food. The chemical evaluation method is a method for performing evaluation by measuring the amount, concentration, or the like of histamine, trimethylamine (TMA), a nucleic acid-related compound, or the like. The physical evaluation method is a method for performing evaluation by measuring the rigor index, texture, impedance, or the like of a perishable food.

As an example of the chemical evaluation method, a method using, as a freshness evaluation index, a K value for performing evaluation using the degradation degree of adenosine triphosphate (ATP) that is a nucleic acid-related compound as an electrophoresis index is known.

As the method for evaluating the freshness of a perishable food, a sensory evaluation method is mainly used. However, the sensory evaluation method relies on the subjectivity of an evaluator, and therefore, quantitative evaluation is not easy. In addition, in electrophoresis or the like using a K value that is a chemical evaluation method, a sample needs to be collected from a food to perform an analysis. Moreover, in electrophoresis or the like using a K value, an expensive analytical apparatus also needs to be used.

Further, there is also a method using a fluorescence reaction as a freshness index, however, a fluorescent state is evaluated by visual observation, and therefore, quantitative evaluation is not easy.

### DESCRIPTION OF THE DRAWINGS

FIG. 1 is a view showing one example of a configuration of a freshness measurement system according to a first embodiment.

FIG. 2 is a view showing a degradation process of a nucleic acid-related substance.

FIG. 3 is a plan view of a phosphor unit in FIG. 1.

FIG. 4 is a perspective view schematically showing a fluorescent structure in FIG. 3.

FIG. 5 is an enlarged cross-sectional view of the fluorescent structure shown in FIG. 4.

FIG. 6 is a view for illustrating a quenching mechanism of an aggregation-induced phosphor.

FIG. 7 is a view summarizing images obtained in Example using acetic acid as a subject component.

FIG. 8 is a view summarizing images obtained in Example using trimethylamine as a subject component.

FIG. 9 is a graph showing one example of a relationship between the storage time of a saurel and the fluorescence intensity of a phosphor unit or the K value.

FIG. 10 is a view including a cross-sectional view taken along the line A-A of FIG. 1, and a view for illustrating a light sensor in FIG. 1.

FIG. 11 is a view including a cross-sectional view taken along the line A-A of FIG. 1, and a view for illustrating a light sensor in FIG. 1.

FIG. 12 is a block diagram showing one example or the like of a configuration of a main circuit of a processing unit in FIG. 1.

FIG. 13 is a flowchart showing one example of processing by a processor in FIG. 12.

FIG. 14 is a view showing one example of a measurement screen displayed on a display device in FIG. 1.

FIG. 15 is a view showing one example of a result screen displayed on a display device in FIG. 1.

FIG. 16 is a view showing one example of a configuration of a freshness measurement system according to a second embodiment.

FIG. 17 is a view showing a modification example of an arrangement of an excitation light source and a light sensor.

### DETAILED DESCRIPTION

An object to be achieved by embodiments is to provide a freshness measurement system capable of quantitatively evaluating the freshness of a perishable food by a simpler method than a related art without damaging or degrading the object evaluated by the freshness measurement system.

A freshness measurement system according to an embodiment includes an irradiation unit, a measurement unit, and a processing unit. The irradiation unit irradiates a phosphor that changes the intensity of fluorescence according to the concentration of a component released from a test subject with an excitation light. The measurement unit measures the intensity of fluorescence emitted by the phosphor. The processing unit determines the freshness index of the test subject using the intensity.

Hereinafter, the freshness measurement system according to embodiments will be described with reference to the drawings. Note that in the respective drawings used in the description of the following embodiments, the scale for each member is sometimes changed as appropriate. Further, the respective drawings used in the description of the following embodiments are sometimes shown by omitting components for the sake of explanation. In addition, in the respective drawings and the present specification, the same reference numerals denote the same elements.

### First Embodiment

FIG. 1 is a view showing one example of a configuration of a freshness measurement system 1 according to a first embodiment. In one example, the freshness measurement system 1 includes a measurement device 100, a measurement container 200, a phosphor unit 300, a processing device 400, and a display device 500.

The measurement device 100 measures or gauges the fluorescence intensity of a fluorescent structure 320 included in the phosphor unit 300. In one example, the measurement device 100 includes a control circuit 110, an excitation light source 120, a light sensor 130, a cut-off filter 140, an analog-to-digital converter (ADC) 150, a processing I/F 160, and a power supply 170.

The control circuit 110 is, for example, a circuit board including a drive circuit for driving the excitation light source 120, or the like. Further, the control circuit 110 is a circuit board including, for example, a wiring for connecting the respective members of the measurement device 100. Further, the control circuit 110 is, for example, a circuit board including an integrated circuit (IC) to be used for controlling the respective members of the measurement device 100. Further, the control circuit 110 may include at least either of an amplifier and an AD conversion circuit as needed. The AD conversion circuit converts an analog signal output from the light sensor 130 to a digital signal. The amplifier amplifies a signal output from the light sensor 130.

The excitation light source 120 is a light source that emits a light for exciting the fluorescent structure 320. Typically, the excitation light source 120 mainly emits an ultraviolet light. Note that within the scope of the present specification and claims, the “light” shall also include a light with a wavelength outside the visible light region (electromagnetic waves). Accordingly, the excitation light source 120 is an example of the irradiation unit that irradiates a phosphor with an excitation light.

The light sensor 130 is a sensor for measuring the fluorescence intensity of the fluorescent structure 320. The light sensor 130 is a sensor capable of measuring the intensity of a light with the same wavelength as that of fluorescence emitted by the fluorescent structure 320. The light sensor 130 is, for example, a photodiode, a photoresistor, a phototransistor, a phototube, a photoconductive cell, a photovoltaic cell, a camera, or the like. When the light sensor 130 is a camera, the camera includes, for example, an image sensor such as a charge-coupled device (CCD) image sensor, or a complementary metal-oxide semiconductor (CMOS) image sensor. Note that the light sensor 130 outputs, for example, a signal based on a light intensity [watt (W)] or a luminosity factor such as a luminous intensity, a luminance, or an illuminance as the intensity of the light. Further, when the light sensor 130 is a camera, the light sensor 130 outputs, for example, an image signal. Accordingly, the light sensor 130 is one example of the measurement unit that measures the intensity of fluorescence emitted by the fluorescent structure 320.

The cut-off filter 140 is a filter for cutting off a light in a wavelength band of a light emitted by the excitation light source 120. The cut-off filter 140 is typically an ultraviolet (UV) cut-off filter for cutting off a light in a wavelength region including an ultraviolet light. The cut-off filter 140 prevents a light emitted by the excitation light source 120 from being incident on the light sensor 130 by cutting off an ultraviolet light.

The ADC 150 converts an input analog signal to a digital signal and outputs the digital signal. Note that the ADC 150 may be built in the light sensor 130.

The processing I/F 160 outputs a signal output by the ADC 150 to the processing device 400.

The power supply 170 supplies electric power to the respective members of the measurement device 100.

In one example, the measurement container 200 includes a tray 210 and a film 220. Further, the measurement container 200 is used for putting a food 230 or the like therein.

The tray 210 is, for example, a food tray for placing the food 230 or the like thereon.

The film 220 is a film for wrapping the tray 210 on which the food 230 is placed. The measurement container 200 wrapped with the film 220 becomes in a sealed state.

The food 230 is, for example, a perishable food such as meat or fish. Alternatively, the food 230 may be another marine product, livestock product, or the like. The food 230 is a test subject for which the freshness is measured.

The food 230 releases a specific chemical component (hereinafter referred to as a subject component) as the freshness decreases. By using the concentration of the subject component, the freshness of the food 230 can be evaluated. The subject component is, for example, an organic acid and an amine compound, or the like.

Here, the subject component will be described in detail. A perishable food can release one type or a plurality of types of subject components into a gas phase when the food is rotten or deteriorated. Examples of the subject component include acidic components such as aldehydes and carboxylic acids, basic components such as alcohols, ammonia, and amines, esters, and ketones. Examples of the aldehydes include hexanal, 3-methyl butanal, nonanal, and isovaleric aldehyde. Examples of the carboxylic acids include formic acid, acetic acid, isovaleric acid, and mixtures thereof. Examples of the amines include trimethylamine, dimethylamine, 1,2-ethylenediamine, 1,3-propanediamine, 1,4-butanediamine, 1,5-pentanediamine, 1,6-hexanediamine, spermidine, spermine, histamine, tryptamine, and mixtures thereof. Examples of the alcohols include ethanol, isopropyl alcohol, 3-methyl-1-butanol, 1-pentanol, 1-butanol, and mixtures thereof. Examples of the esters include ethyl acetate, methyl acetate, and ethyl propionate. Examples of the ketones include methyl ethyl ketone, acetone, and mercaptoacetone.

Further, adenosine triphosphate (ATP) contained in a perishable food such as the food 230 is degraded as shown in FIG. 2 as follows: from ATP to adenosine diphosphate (ADP), from ADP to adenosine monophosphate (AMP), from AMP to inosine monophosphate (IMP), from IMP to inosine (hypoxanthine riboside (HxR)), and from HxR to hypoxanthine (Hx). ATP, ADP, AMP, and IMP are substances contained in a food with high freshness, and HxR and Hx are substances contained in a food with low freshness. A freshness index using this is a K value. The K value can be represented by, for example, the following formula (A).

K=((HxR+Hx)/(ATP+ADP+AMP+IMP+HxR+Hx))×100[%] (A)

That is, the K value is a value representing a ratio of the total amount of HxR and Hx to the total amount of ATP, ADP, AMP, IMP, HxR, and Hx in percentage. Note that the higher the K value is, the lower the freshness is, and the lower the K value is, the higher the freshness is. The amounts of ATP, ADP, AMP, IMP, HxR, and Hx in a food are calculated by, for example, high performance liquid chromatography or electrophoresis. In general, when the K value is 60% or more, the food is determined to be rotten. The evaluation based on the K value can relatively accurately indicate the freshness of a food.

FIG. 3 is a plan view of the phosphor unit 300. In one example, the phosphor unit 300 includes a base part 310, a fluorescent structure 320, a white standard plate 330, and a black standard plate 340. The phosphor unit 300 includes the base part 310, the fluorescent structure 320, and the white standard plate 330.

To the base part 310, the fluorescent structure 320, the white standard plate 330, and the black standard plate 340 are attached. The material of the base part 310 is preferably a material having water resistance, acid resistance, and alkali resistance. Further, a material that causes no emission of fluorescence of the base part 310 itself is preferred, however, the material is not particularly limited as long as the material does not have an effect when the fluorescence of the fluorescent structure 320 is measured even if the material emits fluorescence. The base part 310 is, for example, a transparent sheet-like resin or the like.

The fluorescent structure 320 reacts with the subject component generated from the food 230 so that the intensity of fluorescence is changed. For example, as the concentration of the subject component in a gas G in the measurement container 200 is higher, the intensity of fluorescence of the fluorescent structure 320 becomes low. That is, the fluorescent structure 320 indicates that as the intensity of fluorescence is higher, the freshness of the food 230 is higher, and as the intensity of fluorescence is lower, the freshness of the food 230 is lower. Alternatively, the fluorescent structure 320 may be configured such that as the concentration of the subject component in the gas G is higher, the intensity of fluorescence becomes higher. That is, the fluorescent structure 320 may be configured to indicate that as the intensity of fluorescence is higher, the freshness of the food 230 is lower, and as the intensity of fluorescence is lower, the freshness of the food 230 is higher. Note that the gas G may be a mixed gas containing the subject component or may be an aerosol containing the subject component as a dispersoid.

The fluorescent structure 320 is one example of the phosphor that changes the intensity of fluorescence according to the concentration of the subject component released from the test subject.

Hereinafter, one example of the fluorescent structure 320 will be described.

FIG. 4 is a perspective view schematically showing the fluorescent structure according to the embodiment. The fluorescent structure 320 shown in FIG. 4 includes a base material 321 and a phosphor layer (not shown). FIG. 5 is an enlarged cross-sectional view of the fluorescent structure shown in FIG. 4. The fluorescent structure 320 shown in FIGS. 4 and 5 is an example using a filter paper as the base material 321. On a fiber 321a of the base material 321, a phosphor layer 322 is carried. The phosphor layer 322 includes an aggregation-induced phosphor 322a adhered to the fiber 321a of the base material 321.

The shape, material, and the like of the base material 321 are not limited as long as the base material can be impregnated with water. The base material 321 is, for example, a porous material or a mesh structure. The shape of the base material 321 may be a circular shape as shown in FIG. 4 or may be a polygonal shape. The thickness of the base material 321 is, for example, 0.1 mm or more and 1.0 mm or less. The thickness is not particularly limited as long as the amount of fluorescence emitted from the aggregation-induced phosphor can be ensured, and may be such a thickness that the base material does not inhibit the reaction of the aggregation-induced phosphor with a rotten component inside the base material due to too much thickness on the contrary.

The base material 321 contains, for example, a synthetic fiber, an inorganic fiber, a natural fiber, or a mixture thereof. Examples of the synthetic fiber include a polyolefin-based fiber and a cellulose-based fiber. Examples of the inorganic fiber include a glass fiber, a metal fiber, an alumina fiber, and an active carbon fiber. Examples of the natural fiber include wood pulp and hemp pulp. The base material 321 is preferably a layer composed of a glass fiber.

The phosphor layer 322 contains the aggregation-induced phosphor 322a, and is preferably composed only of the aggregation-induced phosphor 322a. The phosphor layer 322 is carried on the base material 321. The phosphor layer 322 is preferably carried in a thin layer state on the surface of the fiber 321a or the like of the base material 321.

The thickness of the phosphor layer 322 is preferably such a thickness that the fluorescence intensity thereof is sufficiently decreased by leaving the layer in an environment at 25° C. and a relative humidity of 100%. Here, the phrase “the fluorescence intensity is sufficiently decreased” refers to, for example, that when the fluorescence intensity in the case of leaving the layer in an environment at 25° C. and a relative humidity of 100% is calculated as a relative value by assuming the fluorescence intensity in the case of leaving the layer in an environment at 10° C. and a relative humidity of 20% to be 100%, the relative value becomes 30% or less.

The thickness of the phosphor layer 322 can affect the fluorescence intensity of the fluorescent structure 320. That is, when the phosphor layer 322 is moderately thickened, the fluorescence intensity of the fluorescent structure 320 tends to increase. On the other hand, when the phosphor layer 322 is excessively thickened, the change in the fluorescence intensity according to the change in the freshness becomes small. The thickness of the phosphor layer 322 is preferably 30 nm or less, more preferably 20 nm or less. The thickness of the phosphor layer 322 is desirably adjusted within a range in which the change in the freshness of a perishable food is easily confirmed according to the release amount of a subject component to be released due to rot or deterioration of the perishable food. The thickness of the phosphor layer 322 can be confirmed by, for example, transmission electron microscopy (TEM).

The aggregation-induced phosphor 322a may form the phosphor layer 322 as a particulate layer as shown in FIG. 5, or may form the phosphor layer 322 as a continuous membrane with no gaps. In the phosphor layer 322 as a granular layer, each particle contains a plurality of molecules of the aggregation-induced phosphor 322a, and the number of molecules of the aggregation-induced phosphor 322a located on a shortest straight line connecting the surface of the particle from each position in the particle is, for example, 10 or less.

The aggregation-induced phosphor 322a preferably has a polar functional group. The aggregation-induced phosphor 322a containing a polar functional group easily reacts with a subject component so that the accuracy of the freshness evaluation using the phosphor unit can be enhanced. Further, the solubility or the dispersibility in water tends to be high. The polar functional group may be an acidic functional group ora basic functional group. Examples of the acidic functional group include a carboxyl group and a sulfo group. Examples of the basic functional group include a hydroxy group and an amino group. The aggregation-induced phosphor 322a may contain a plurality of types of acidic functional groups or basic functional groups. The aggregation-induced phosphor 322a preferably contains two or more carboxyl groups in one molecule.

As the aggregation-induced phosphor 322a, one having a tetraphenylethylene skeleton represented by a structural formula (2), a silole skeleton represented by a structural formula (3), or a phosphole oxide skeleton represented by a structural formula (4) can be used. Each of these compounds may be a cis form or a trans form, or may be a mixture of a cis form and a trans form.

[chemical structure]

The aggregation-induced phosphor 322a preferably contains a tetraphenylethylene derivative represented by the following general formula (I). The compound represented by the following general formula (I) has excellent reactivity with a subject component.

[chemical structure]

(In the formula, R₁, R₂, R₃, and R₄ are mutually independently selected from the group consisting of -L₁M₁, —(CH₂)ₘ-L₂M₂, —X— (CH₂)ₙ-L₃M₃, —Y—(CH₂)ₒ—Z— (CH₂)ₚ-L₄M₄ (wherein L₁, L₂, L₃, and L₄ mutually independently represent —CO₂— or —SO₃—, M₁, M₂, M₃, and M₄ mutually independently represent a hydrogen atom or a cation, X, Y, and Z mutually independently represent —O—, —NH—, or —S—, m, n, o, and p mutually independently represent an integer of 1 to 6), a hydrogen atom, a halogen atom, a hydroxy group, a nitro group, a carbamoyl group, an alkyl group having 1 to 6 carbon atoms, a haloalkyl group having 1 to 6 carbon atoms, an alkenyl group having 2 to 6 carbon atoms, a cycloalkyl group having 3 to 10 carbon atoms, an alkyloxy group having 1 to 6 carbon atoms, an acyl group having 2 to 6 carbon atoms, an amino group, an alkylamino group having 1 to 6 carbon atoms, an aryl group having 6 to 10 carbon atoms, and a heteroaryl group having 5 to 10 carbon atoms, and at least two of R₁, R₂, R₃, and R₄ are mutually independently selected from the group consisting of -L₁M₁, —(CH₂)ₘ-L₂M₂, —X— (CH₂)ₙ-L₃M₃, and —Y— (CH₂)ₒ—Z— (CH₂)ₚ-L₄M₄ (wherein L₁, L₂, L₃, L₄, M₁, M₂, M₃, M₄, X, Y, Z, m, n, o, and p are as described above.)

Specific examples of the tetraphenylethylene derivative include compounds represented by the following structural formulae (5), (7), (9), and (10).

[chemical structure]

The fluorescent structure 320 is produced by, for example, the following method.

First, a processing liquid is prepared by dissolving the aggregation-induced phosphor 322a in an organic solvent. The type of the organic solvent may be any as long as the solvent can dissolve the aggregation-induced phosphor 322a, and a solvent having a low evaporation temperature is preferred. As the organic solvent, for example, ethanol is used. The concentration of the aggregation-induced phosphor 322a in the processing liquid is set to, for example, 50 μm (molar weight) or more and 1 mM (molar weight) or less when using the compound represented by the above structural formula (7).

Subsequently, the base material 321 is immersed in the processing liquid and impregnated with the processing liquid, and then, the base material 321 is pulled up from the processing liquid, followed by drying. Note that the base material 321 may be impregnated with the processing liquid by dropping the processing liquid using a Pasteur pipette or the like. In this manner, the fluorescent structure 320 is obtained. The fluorescent structure 320 typically does not contain an organic solvent.

The white standard plate 330 and the black standard plate 340 each become a standard for the measurement of the fluorescence intensity of the fluorescent structure 320.

The white standard plate 330 is a white plate, sheet or the like that diffuses and reflects a light. The white standard plate 330 preferably has a reflectance as high as possible. An ideal white standard plate 330 has a reflectance of 100%.

The black standard plate 340 is a black plate, sheet, or the like. The black standard plate 340 has a reflectance as low as possible. An ideal black standard plate 340 has a reflectance of 0%.

Further, the phosphor unit 300 retains water. For example, the fluorescent structure 320 retains water. Alternatively, the fluorescent structure 320 and the base part 310 retain water. In the phosphor unit 300, the mass of water with respect to the mass of the aggregation-induced phosphor 322a is, for example, 0.5 or more.

In the production of the phosphor unit 300, first, the fluorescent structure 320 is immersed in water and impregnated with water, and then, pulled up from water. Note that the fluorescent structure 320 may be impregnated with water by dropping water using a Pasteur pipette or the like, or the fluorescent structure 320 may be exposed to water vapor to incorporate water. As the type of water, distilled water, pure water, ion exchanged water, or a mixture thereof can be used.

The fluorescent structure 320 pulled up from water is attached to the base part 310 using a joining member such as an adhesive or an adhesive tape. Alternatively, a cut or a through-hole is provided in a portion of the base part 310, and the fluorescent structure 320 may be fixed by being fitted in the cut or the through-hole. In this manner, the phosphor unit 300 is obtained. Note that the phosphor unit 300 may be obtained by integrating the fluorescent structure 320 and the base part 310, and thereafter immersing the integrated material in water.

As described above, the phosphor unit 300 according to the embodiment contains water carried by the fluorescent structure 320 and the base material 321. The content amount of water may be an amount that makes the fluorescence of the phosphor unit 300 weak or null. That is, the fluorescent structure 320 exhibits weak fluorescence or no fluorescence by incorporating water.

FIGS. 10 and 11 are each a view including a cross-sectional view taken along the line A-A of FIG. 1, and a view for illustrating the light sensor 130. Note that the cross-sectional view taken along the line A-A of FIG. 1 is a cross-sectional view of the measurement container 200 and the phosphor unit 300. Further, FIGS. 10 and 11 show the cross-sectional view taken along the line A-A of FIG. 1 by omitting a portion. In FIGS. 10 and 11, a subject component (X) is also shown.

As shown in FIGS. 10 and 11, the phosphor unit 300 is adhered to the film 220 so that the fluorescent structure 320 faces the side where the food 230 is present. This is because the fluorescent structure 320 needs to be exposed to (come into contact with) a gas G in the measurement container 200 so as to come into contact with the subject component X. Further, as shown in FIGS. 10 and 11, the white standard plate 330 and the black standard plate 340 are preferably located on the opposite side of the fluorescent structure 320 across the base part 310. That is, the white standard plate 330 and the black standard plate 340 are preferably located on the opposite side of the food 230 across the base part 310. This is because the white standard plate 330 and the black standard plate 340 are prevented from being exposed to the gas G. According to this, the white standard plate 330 and the black standard plate 340 are prevented from being denatured and fouled by being exposed to the gas G.

Note that the phosphor unit 300 may be in a state of being placed in the measurement container 200 without being adhered to the film 220. However, the phosphor unit 300 shall be placed therein so that the phosphor layer 322 can be seen from the outside of the measurement container 200.

The phosphor unit 300 can quantitatively determine the freshness of the food 230 by irradiation with an excitation light such as an ultraviolet light and measuring the intensity (brightness) of fluorescence thereof. Here, as one example, the subject component generated with the deterioration of the freshness of the food 230 shall be an acidic component, and the aggregation-induced phosphor 322a shall contain an acidic functional group as the polar functional group.

When the food 230 is in a fresh state, the concentration of the subject component in the gas G is low. In that case, the effect of the subject component on the arrangement of the molecules of the aggregation-induced phosphor 322a is small. Therefore, in that case, even if the aggregation-induced phosphor 322a is irradiated with an excitation light, the aggregation-induced phosphor 322a does not emit fluorescence with a high intensity.

When the freshness of the food 230 decreases, the concentration of the subject component in the gas G increases. When the concentration of the subject component in the gas G increases, a portion thereof is dissolved in water contained in the phosphor unit 300. This aqueous solution has the same polarity as the polar functional group of the aggregation-induced phosphor 322a, and therefore, when the concentration of the subject component in the aqueous solution increases, the affinity or the solubility of the aggregation-induced phosphor 322a in the aqueous solution decreases. Therefore, when the concentration of the subject component in the gas G increases, the molecular arrangement of the aggregation-induced phosphor 322a approaches a state where water is not present. Accordingly, it is considered that when the freshness of the food 230 decreases, the intensity of fluorescence emitted by the aggregation-induced phosphor 322a by irradiation with an excitation light increases.

In the irradiation of the phosphor unit 300 with an excitation light, for example, an ultraviolet (UV) lamp is used. The wavelength of the ultraviolet light varies depending on the type of the aggregation-induced phosphor 322a, but is 350 nm or more and 530 nm or less in one example. Further, in the measurement of the fluorescence intensity, for example, a light detector or an image capture element is used as described above. For example, first, a fluorescence image is captured using a digital camera or the like while irradiating the phosphor unit 300 with a UV lamp.

Note that when as the aggregation-induced phosphor 322a, one having an acidic functional group is used, and the subject component is an acidic component as described above, the fluorescence intensity of the phosphor unit 300 further increases with the increase in the concentration of the subject component.

As described above, when the phosphor unit 300 is used, by a simple method in which the phosphor unit 300 is irradiated with an excitation light such as an ultraviolet light, and the intensity (brightness) of fluorescence thereof is measured, the freshness of the food 230 can be quantitatively determined. Moreover, this quantitative determination of the freshness can be performed with high accuracy.

Next, another method for using the phosphor unit 300 according to the embodiment will be described. The phosphor unit 300 may further contain an acid. The phosphor unit 300 further containing an acid is hereinafter referred to as a phosphor unit 300A. The phosphor unit 300A has the same structure as the phosphor unit 300 that does not further contain an acid except for further containing an acid. That is, the phosphor unit 300A includes the fluorescent structure 320, the base part 310, and water and an acid (not shown). The fluorescent structure 320 carries water and an acid (not shown). In other words, the fluorescent structure 320 carries an acidic aqueous solution. As the acid, for example, formic acid, hydrochloric acid, acetic acid, or a mixture thereof is used. As the acid, acetic acid is preferably used from the safety viewpoint. Further, the polar functional group of the aggregation-induced phosphor 322a is preferably an acidic functional group. Here, as one example, the polar functional group of the aggregation-induced phosphor 322a shall be an acidic functional group.

The phosphor unit 300A is obtained by, for example, exposing the phosphor unit 300 that does not further contain an acid to a vapor containing an acid at a high concentration. The content amount of the acid in the phosphor unit 300A can be adjusted according to a desired fluorescence intensity.

Since the phosphor unit 300A contains an acid, it is considered that the molecular arrangement of the aggregation-induced phosphor 322a is close to a state where water is not contained, or the conformation of the aggregation-induced phosphor 322a is changed or the like due to the presence of the acid component. Therefore, the phosphor unit 300A exhibits stronger fluorescence than the phosphor unit 300 that does not further contain an acid.

The fluorescence intensity of the phosphor unit 300A decreases when the phosphor unit 300A comes into contact with a basic subject component, and when the phosphor unit 300A comes into contact with a certain amount or more of the basic subject component, no fluorescence is exhibited. That is, the fluorescence intensity of the phosphor unit 300A can show a negative correlation with the concentration of the subject component and the K value. Therefore, in the same manner as the phosphor unit 300 that does not further contain an acid, when the fluorescence intensity of the phosphor unit 300A is converted to a numerical value, and a calibration curve representing the relationship between the numerical value and the concentration of the subject component or the K value is prepared in advance, the concentration of the subject component in the gas phase or the K value of the food can be obtained by measuring the fluorescence intensity and referring the measurement result to the calibration curve.

The phosphor unit 300 may be distributed in a state where water or the like is not contained, and water or the like may be incorporated, for example, at a site where the phosphor unit 300 is enclosed in an airtight container together with the food 230. In that case, the phosphor unit 300 may be distributed as a phosphor unit kit including the phosphor unit 300 and one or more liquids to be incorporated therein, that is, water, an acid, or a combination of water and an acid. When the combination of water and an acid is included in the phosphor unit kit, water and the acid may be stored in separate containers, or may be mixed and stored in a single container as an aqueous solution.

Alternatively, a phosphor unit obtained by incorporating water or the like in the phosphor unit 300 is distributed and may be enclosed in an airtight container together with the food 230.

The fluorescent structure according to the embodiment described above includes the phosphor layer adhered to the base material. Therefore, by impregnating the fluorescent structure with water, the phosphor unit can be prepared. The phosphor unit containing water is hardly affected by water in the gas phase, and therefore, can evaluate the freshness of a food with higher accuracy as compared with a phosphor unit containing an organic solvent.

Examples of the phosphor unit 300 will be described below.

1. Example Using Acetic Acid as Subject Component

The phosphor unit 300 was prepared by the following method. First, the aggregation-induced phosphor 322a was dissolved in ethanol, whereby a processing liquid was prepared. The base material 321 was immersed in the processing liquid, and thereafter, the base material 321 was pulled up from the processing liquid, and then placed on a glass plate and dried. As the aggregation-induced phosphor 322a, a compound represented by the above-mentioned structural formula (5) was used. As the base material 321, a circular glass fiber filter was used. In this manner, the fluorescent structure 320 shown in FIG. 4 was obtained. In the fluorescent structure 320, when the phosphor layer 322 provided on the surface of the glass fiber was observed with TEM, the thickness thereof was 20 nm. When the fluorescent structure 320 was observed while being irradiated with an ultraviolet light using a UV lamp, it was confirmed that strong fluorescence was emitted. The wavelength of the ultraviolet light irradiated using the UV lamp was 365 nm. Further, an image of the fluorescent structure 320 at that time was captured using a digital camera, and the digital image data were recorded.

Subsequently, the fluorescent structure 320 was immersed in pure water, and then, pulled up from pure water, whereby water was impregnated into the base material 321. In this manner, the phosphor unit 300 was obtained. The base part 310 was omitted. Hereinafter, this phosphor unit is referred to as a phosphor unit R1. When the phosphor unit R1 was observed while being irradiated with an ultraviolet light using a UV lamp, it was confirmed that no fluorescence was emitted. Further, an image of the phosphor unit R1 at that time was captured using a digital camera, and the digital image data were recorded.

Subsequently, two sheets of the phosphor unit R1 were disposed in a container in which a vapor of an acetic acid aqueous solution was sealed and a container in which a vapor of pure water was sealed, respectively, and exposed to the vapor of the acetic acid aqueous solution and the vapor of pure water, respectively. As the acetic acid aqueous solution, a mixed solution of 20 μL of acetic acid and 200 μL of water was used. Further, in both the airtight containers, 1 g of pure water was stored for keeping moisture. As the container, an airtight container made of a plastic and having a volume of about 100 mL was used. In the container, the phosphor unit R1 in a circular shape with a diameter of 21 mm was disposed at a distance from the acetic acid aqueous solution or water so as to prevent direct wetting thereof.

After the elapse of 1 hour, the phosphor unit R1 was taken out from each container, and observed while being irradiated with an ultraviolet light using a UV lamp, and the fluorescence intensity was confirmed. As a result, the phosphor unit R1 disposed in the container in which the vapor of the acetic acid aqueous solution was sealed exhibited a fluorescence intensity equivalent to that of the fluorescent structure 320. On the other hand, in the phosphor unit R1 disposed in the container in which the vapor of pure water was sealed, the fluorescence remained null. In addition, an image of the phosphor unit R1 at that time was captured using a digital camera, and the digital image data were recorded.

FIG. 7 is a table summarizing the images obtained in Example using acetic acid as the subject component. From FIG. 7, it is found that the fluorescence intensity of the phosphor unit R1 is only affected by the acetic acid aqueous solution without being affected by pure water. Therefore, when using the phosphor unit R1, the freshness can be quantitatively determined with high accuracy in the case of using acetic acid as the subject component.

2. Example Using Trimethylamine as Subject Component

First, the phosphor unit R1 was produced in the same manner as described above. The phosphor unit R1 was enclosed in a container in which a vapor of an acetic acid aqueous solution was sealed, and exposed to the vapor of the acetic acid aqueous solution over a sufficient time, whereby the phosphor unit 300A was obtained. Hereinafter, the phosphor unit 300A is referred to as a phosphor unit R2. As the acetic acid aqueous solution, a mixed solution of 20 μL of acetic acid and 200 μL of water was used. Further, in the airtight container, 1 g of pure water was stored for keeping moisture. When the phosphor unit R2 was observed while being irradiated with an ultraviolet light using a UV lamp, it was confirmed that strong fluorescence is emitted. In addition, an image of the phosphor unit R2 at that time was captured using a digital camera, and the digital image data were recorded.

Subsequently, two sheets of the phosphor unit R2 were disposed in a container in which a vapor of a trimethylamine aqueous solution was sealed and a container in which a vapor of pure water was sealed, respectively, and exposed to the vapor of the trimethylamine aqueous solution and the vapor of pure water, respectively. As the trimethylamine aqueous solution, a mixed solution containing 10 μL of trimethylamine, 30 μL of ethanol, and 160 μL of water was used. Further, in both the airtight containers, 1 g of pure water was stored for keeping moisture.

After the elapse of 1 hour, the phosphor unit R2 was taken out from each container, and observed while being irradiated with an ultraviolet light using a UV lamp, and the fluorescence intensity was confirmed. As a result, the fluorescence intensity of the phosphor unit R2 disposed in the container in which the vapor of the trimethylamine aqueous solution was sealed significantly decreased as compared with that before the test. On the other hand, the fluorescence intensity of the phosphor unit R2 disposed in the container in which the vapor of pure water was sealed was substantially equivalent to that before the test. In addition, an image of the phosphor unit R2 at that time was captured using a digital camera and recorded using the digital image data.

FIG. 8 is a table summarizing the images obtained in Example using trimethylamine as the subject component. From FIG. 8, it is found that the fluorescence intensity of the phosphor unit R2 is only affected by the trimethylamine aqueous solution without being affected by pure water. Therefore, when using the phosphor unit R2, the freshness can be quantitatively determined with high accuracy in the case of using trimethylamine as the subject component.

3. Example Using Fresh Fish

First, the measurement container 200 in which the food 230 was put was prepared. As the food 230, a saurel was used. As the phosphor unit 300, the above phosphor unit R2 was used. The measurement container 200 was stored in an environment at 25° C., and the fluorescence intensity of the phosphor unit R2 and the K value were measured for each storage time shown in the following Table 1. The fluorescence intensity was converted to a numerical value by calculating the gradation value of RGB using an image processing software based on the image data obtained when irradiation with an ultraviolet light using a UV lamp, and determining an arithmetic average thereof. Further, the K value was determined using electrophoresis by collecting a portion of the saurel. The results are shown in FIG. 9. In the electrophoresis, the measurement was performed using a freshness checker manufactured by QS-Solution.

FIG. 9 is a graph showing one example of a relationship between the storage time of the saurel and the fluorescence intensity of the phosphor unit or the K value. As shown in FIG. 9, a high correlation was observed between the fluorescence intensity of the phosphor unit and the K value. Therefore, when the phosphor unit R2 is used, the freshness of a saurel can be evaluated with high accuracy. Note that when in a saurel sample used in the test, an odor component was confirmed using gas chromatography, trimethylamine was detected from the sample in which the freshness of the saurel decreased. Further, by converting the fluorescence intensity of the phosphor unit R2 to a numerical value and generating a calibration curve, an approximate K value can be determined.

The light sensor 130 will be further described with reference to FIGS. 10 and 11.

The light sensor 130 can move, for example, in the horizontal direction H as shown in FIG. 10. The light sensor 130 measures the brightness of each of the fluorescent structure 320, the white standard plate 330, and the black standard plate 340 by moving. Alternatively, the light sensor 130 may be configured to change the direction of measurement by rotation or the like. In that case, the light sensor 130 measures the brightness of each of the fluorescent structure 320, the white standard plate 330, and the black standard plate 340 by changing the direction of measurement.

Alternatively, the measurement device 100 includes three light sensors 130: a light sensor 130-1, a light sensor 130-2, and a light sensor 130-3. The light sensor 130-1 measures the brightness of the fluorescent structure 320. The light sensor 130-2 measures the brightness of the white standard plate 330. The light sensor 130-3 measures the brightness of the black standard plate 340.

Alternatively, when the light sensor 130 is a camera, the measurement device 100 measures the brightness of each of the fluorescent structure 320, the white standard plate 330, and the black standard plate 340 using one fixed light sensor 130.

As described above, the light sensor 130 measures the brightness of each of the fluorescent structure 320, the white standard plate 330, and the black standard plate 340. Note that the brightness of the fluorescent structure 320 indicates the intensity of fluorescence of the fluorescent structure 320.

FIG. 12 is a block diagram showing one example of a configuration of a main circuit of the processing device 400, or the like.

The processing device 400 is, for example, a personal computer (PC). Alternatively, the processing device 400 may be a tablet PC, a smartphone, or the like. In one example, the processing device 400 includes a processor 410, a read-only memory (ROM) 420, a random-access memory (RAM) 430, an auxiliary memory device 440, a measurement I/F 450, a display I/F 460, and an input I/F 470. Then, a bus 480 or the like connects these respective members.

The processor 410 corresponds to a central part of the computer that executes processing such as calculation and control necessary for an operation of the processing device 400. The processor 410 controls the respective members for realizing various functions of the processing device 400 based on a program such as firmware, system software, or application software stored in the ROM 420, the auxiliary memory device 440, or the like. Further, the processor 410 executes the below-mentioned processing based on the program. Note that a part or all of the program may be integrated into the circuit of the processor 410. The processor 410 is, for example, a central processing unit (CPU), a micro processing unit (MPU), a system on a chip (SoC), a digital signal processor (DSP), a graphics processing unit (GPU), an application specific integrated circuit (ASIC), a programmable logic device (PLD), a field-programmable gate array (FPGA), or the like. Alternatively, the processor 410 is configured to combine two or more members thereof.

The ROM 420 corresponds to a main memory device of the computer having the processor 410 as the center. The ROM 420 is a non-volatile memory exclusively used for reading data. The ROM 420 stores, for example, firmware or the like among the above-mentioned programs. Further, the ROM 420 also stores data or the like used for the processor 410 to execute all sorts of processing.

The RAM 430 corresponds to a main memory device of the computer having the processor 410 as the center. The RAM 430 is a memory used for reading and writing data. The RAM 430 is utilized as a work area for storing data temporarily used for the processor 410 to execute all sorts of processing. The RAM 430 is typically a volatile memory.

The auxiliary memory device 440 corresponds to an auxiliary memory device of the computer having the processor 410 as the center. The auxiliary memory device 440 is, for example, an electric erasable programmable read-only memory (EEPROM), a hard disk drive (HDD), a flash memory, or the like. The auxiliary memory device 440 stores, for example, system software, application software, or the like among the above-mentioned programs. Further, the auxiliary memory device 440 stores data used for the processor 410 to execute all sorts of processing, data generated by the processing by the processor 410, all sorts of setting values or the like.

Further, the auxiliary memory device 440 stores freshness measurement application software. The freshness measurement application software is application software used for the operation of the measurement device 100. The freshness measurement application software includes freshness database 441. The freshness database 441 stores and manages all sorts of data necessary for freshness measurement. The freshness database 441 stores data indicating a relationship between the fluorescence intensity and the K value for each type of food. The data are a lookup table (LUT), a function, or the like. Further, the freshness database 441 stores a threshold for being able to eat raw T1 and a threshold for being able to eat T2 for each type of food. The threshold for being able to eat raw T1 and the threshold for being able to eat T2 will be described later.

The measurement I/F 450 is an interface for communicating with the measurement device 100. The measurement I/F 450, for example, receives an input of a signal output from the measurement device 100. Further, the measurement I/F 450, for example, outputs a command or the like to the measurement device 100.

The display I/F 460 is an interface for connecting the processing device 400 to the display device 500.

The display device 500 displays a screen for notifying an operator of the processing device 400 of all sorts of information. The display device 500 is, for example, a display such as a liquid crystal display or an organic electro-luminescence (EL) display.

The input I/F 470 is an interface for connecting the processing device 400 to the input device 600.

The input device 600 accepts an operation from an operator of the processing device 400. The input device 600 is, for example, a keyboard, a keypad, a touch pad, a mouse, or the like. Further, as the display device 500 and the input device 600, a touch panel can also be used. That is, a display panel included in a touch panel can be used as the display device 500. Then, a pointing device operated by a touch input included in the touch panel can be used as the input device 600.

The bus 480 includes a control bus, an address bus, a data bus, and the like, and transmits a signal exchanged by the respective members of the processing device 400.

Hereinafter, an operation of the freshness measurement system 1 according to an embodiment will be described with reference to FIG. 13 or the like. Note that the contents of the processing in the following description of the operation are one example, and various processing capable of obtaining a similar result can be utilized as appropriate. FIG. 13 is a flowchart showing one example of processing by the processor 410 of the processing device 400. The processor 410, for example, executes the processing based on a program stored in the ROM 420, the auxiliary memory device 440, or the like.

The processor 410, for example, executes the freshness measurement application software, and also starts the processing shown in FIG. 13.

The processor 410 in ACT 11 generates an image corresponding to a measurement screen SC1a. Then, the processor 410 instructs the display device 500 via the display I/F 460 to display the generated image. By being instructed to display the image, the display device 500 displays the measurement screen SC1a.

FIG. 14 is a view showing one example of the measurement screen SC1a. In one example, the measurement screen SC1a includes areas AR1 to AR6, and buttons B1 to B3.

In the area AR1, an image IM1 captured by a camera included in the measurement device 100 is displayed. When the measurement device 100 does not include a camera, the measurement screen SC1a does not need to include the area AR1.

The area AR2 is an area for displaying the name of the type of the food as a measurement subject (hereinafter referred to as “food to be measured”) selected. Further, the area AR2 is an area indicating a button or the like for selecting the food to be measured. In the area AR2 shown in FIG. 14, a pull-down menu for selecting the food to be measured is displayed as one example.

The food to be measured is, for example, the type of the food indicating the food 230 that is a subject for which the freshness is measured such as a saury, a horse mackerel, beef, or the like.

The area AR3 displays the intensity of fluorescence measured.

The area AR4 displays the K value measured.

The area AR5 displays the determination result of the state of the food 230.

The area AR6 displays an indicator indicating the intensity of fluorescence and the K value measured. Further, the scale of the indicator also shows a relationship between the fluorescence intensity and the K value corresponding to the food to be measured selected in the area AR2.

The button B1 is a button for an operator of the processing device 400 to perform an operation when changing the setting.

The button B2 is a button for an operator of the processing device 400 to perform an operation when switching on and off of the excitation light source 120. When the button B2 is operated while the excitation light source 120 is turned off, the processor 410 controls the measurement device 100 via the measurement I/F 450 so that the excitation light source 120 is turned on. Further, when the button B2 is operated while the excitation light source 120 is turned on, the processor 410 controls the measurement device 100 via the measurement I/F 450 so that the excitation light source 120 is turned off. By visually observing the image IM1 displayed in the area AR1 while the excitation light source 120 is turned on, the level of fluorescence or the like of the fluorescent structure 320 can be confirmed.

The button B3 is a button for an operator of the processing device 400 to perform an operation when starting the measurement of the freshness of the food 230.

In ACT 12, the processor 410 determines whether to start measurement. The processor 410 determines to start measurement in response to the execution of the operation for starting measurement such as an operation of the button B3. When the processor 410 does not determine to start measurement, the determination is made as “No” in ACT 12, and the process proceeds to ACT 13.

In ACT 13, the processor 410 determines whether or not the type of the food to be measured was selected. The processor 410, for example, determines that the type of the food to be measured was selected in response to the execution of the selection of the food to be measured by the operation for the area AR2. When the processor 410 does not determine that the type of the food to be measured was selected, the determination is made as “No” in ACT 13, and the process returns to ACT 12. In such a manner, the processor 410 repeats ACT 12 and ACT 13 until the processor 410 determines to start measurement or determines that the type of the food as the measurement subject was selected.

When the type of the food to be measured was selected while the processor 410 is in a standby state of ACT 12 and ACT 13, the determination is made as “Yes” in ACT 13, and the process proceeds to ACT 14.

In ACT 14, the processor 410 acquires data corresponding to the selected food to be measured from the freshness database 441. Further, the processor 410 controls the display device 500 via the display I/F 460 to display the type of the selected food to be measured in the area AR2. In addition, the processor 410 controls the display device 500 via the display I/F 460 to update the scale of the indicator in the area AR6 so as to correspond to the selected food to be measured.

In ACT 15, the processor 410 determines whether or not the fluorescence intensity is already measured. When the fluorescence intensity is not already measured, the processor 410 makes a determination as “No” in ACT 15, and the process returns to ACT 12.

When the processor 410 determines to start measurement while being in a standby state of ACT 12 and ACT 13, the determination is made as “Yes” in ACT 12, and the process proceeds to ACT 16.

In ACT 16, the processor 410 controls the measurement device 100 via the measurement I/F 450 to turn on the excitation light source 120. Based on the control, the control circuit 110 of the measurement device 100 turns on the excitation light source 120. By the turning on of the excitation light source 120, the fluorescent structure 320 emits fluorescence.

In Act 17, the processor 410 controls the measurement device 100 via the measurement I/F 450 to measure the brightness of each of the fluorescent structure 320, the white standard plate 330, and the black standard plate 340. Based on the control, the control circuit 110 of the measurement device 100 measures the brightness of each of the fluorescent structure 320, the white standard plate 330, and the black standard plate 340 using the light sensor 130. The light sensor 130 outputs a signal indicating the measurement result. The signal is input to the processing device 400 via the ADC 150, the processing I/F 160, and the measurement I/F 450.

Note that when the light sensor 130 is a camera, the signal input to the processing device 400 is, for example, an image signal. In that case, the processor 410 determines the brightness of each of the fluorescent structure 320, the white standard plate 330, and the black standard plate 340 based on the gradation value of brightness, luminance, or the like with respect to each of the fluorescent structure 320, the white standard plate 330, and the black standard plate 340 from the image. Note that when the light sensor 130 is a camera using a color-type image capture element, the processor 410 determines the intensity of fluorescence as, for example, an average or a weighted average of the gradation value of the brightness of a color component such as red, green, and blue (RGB).

In ACT 18, the processor 410 controls the measurement device 100 via the measurement I/F 450 to turn off the excitation light source 120. Based on the control, the control circuit 110 of the measurement device 100 turns off the excitation light source 120.

In ACT 19, the processor 410 determines a fluorescence intensity ratio Ip [%] as the fluorescence intensity of the fluorescent structure 320 from the measurement value of the brightness of each of the fluorescent structure 320, the white standard plate 330, and the black standard plate 340. For example, the processor 410 determines the fluorescence intensity ratio Ip according to the following formula (B). The fluorescence intensity ratio Ip is one example of a value indicating the intensity of fluorescence.

Ip=(Sp−Sk)/(Sw−Sk) (B)

Sp: a phosphor emission intensity (the measurement value of the brightness of the fluorescent structure 320)

Sk: a white reflection intensity (the measurement value of the brightness of the white standard plate 330)

Sw: a black reflection intensity (the measurement value of the brightness of the black standard plate 340)

In ACT 20, the processor 410 determines a K value from the fluorescence intensity (fluorescence intensity ratio Ip). A relationship between the fluorescence intensity and the K value varies depending on the type of the food to be measured. Therefore, the processor 410 determines the K value based on the data acquired in ACT 14 using, for example, a LUT. Alternatively, the processor 410 may determine the K value using a function or the like.

Accordingly, the processor 410 functions as one example of the processing unit that determines the freshness index of the food 230 by performing the processing of ACT 20.

In ACT 21, the processor 410 makes a determination as to whether the food for which the freshness was measured can be eaten, or the like. For example, the processor 410 determines which is suitable, for example, among “can be eaten raw”, “needs to be heated”, “cannot be eaten”, or the like according to the type of the food to be measured and the K value. Note that the processor 410 does not determine that a food “can be eaten raw” regardless of the freshness depending on the type of the food to be measured. For example, when the K value is less than the threshold for being able to eat raw T1 corresponding to the type of the food to be measured, the processor 410 determines that the food for which the freshness was measured can be eaten raw. Further, when the K value is less than the threshold for being able to eat T2 corresponding to the type of the food to be measured, the processor 410 determines that the food for which the freshness was measured can be eaten. That is, for example, when the K value is less than the threshold for being able to eat raw T1, the processor 410 makes a determination as “can be eaten raw”, and when the K value is not less than the threshold for being able to eat raw T1 but less than the threshold for being able to eat T2, the processor 410 makes a determination as “needs to be heated”, and when the K value is not less than the threshold for being able to eat T2, the processor 410 makes a determination as “cannot be eaten”.

In ACT 22, the processor 410 generates an image corresponding to a result screen SC1b. Then, the processor 410 instructs the display device 500 via the display I/F 460 to display the generated image. By being instructed to display the image, the display device 500 displays the result screen SC1b. After the processing of ACT 22, the processor 410 returns to ACT 12.

FIG. 15 is a view showing one example of the result screen SC1b. The result screen SC1b is configured to display the respective measurement results and the like in addition to the same display as the measurement screen SC1a of FIG. 14.

In the area AR3 in the result screen SC1b, the fluorescence intensity ratio Ip determined in ACT 19 is displayed.

In the area AR4 in the result screen SC1b, the K value determined in ACT 20 is displayed.

In the area AR5 in the result screen SC1b, the determination result in ACT 21 is displayed.

The indicator in the area AR6 in the result screen SC1b indicates the fluorescence intensity ratio Ip determined in ACT 19 and the K value determined in ACT 20.

Accordingly, the display device 500 is one example of a notification unit that makes a notification of the freshness index.

The freshness measurement system 1 of the first embodiment determines the K value of a food by measuring the fluorescence intensity of the fluorescent structure 320 disposed at a position capable of coming into contact with a subject component released from a food that is a measurement subject. According to this, the freshness measurement system 1 of the first embodiment can quantitatively evaluate the freshness. Further, in a related art, in order to calculate the K value, a sample needs to be collected from a food, and also an expensive analytical apparatus needs to be used. On the other hand, according to the freshness measurement system 1 of the first embodiment, a sample does not need to be collected from a food, and further, the K value can be calculated using a simpler and less expensive apparatus than in the related art.

In the freshness measurement system 1 of the first embodiment, the excitation light source 120 is turned on only in the measurement as shown in ACT 16 to ACT 18. Therefore, the freshness measurement system 1 of the first embodiment has a lower operating cost than the case where the excitation light source 120 is kept turned on. Further, the freshness measurement system 1 of the first embodiment prevents deterioration of the fluorescent structure 320 by the effect of the excitation light.

In addition, the freshness measurement system 1 of the first embodiment includes the white standard plate 330 and the black standard plate 340. Then, the freshness measurement system 1 of the first embodiment uses the fluorescence intensity ratio Ip as the fluorescence intensity. Accordingly, the freshness measurement system 1 of the first embodiment can increase the S/N ratio of the fluorescence intensity.

In addition, the freshness measurement system 1 of the first embodiment performs processing of ACT 20 to ACT 22 based on data according to the type of the food after changing when the type of the food is changed after measuring the fluorescence intensity. In this manner, the freshness measurement system 1 of the first embodiment displays the measurement result according to the type of the food after changing when the type of the food is changed afterward, and therefore is easy to use by a user.

### Second Embodiment

A second embodiment is an embodiment using a measurement container including a structure for installing a phosphor unit.

FIG. 16 is a view showing one example of a configuration of a freshness measurement system 1b according to the second embodiment. The freshness measurement system 1b of the second embodiment includes a measurement device 100, a phosphor unit 301, a processing device 400, a display device 500, an input device 600, and a measurement container 700. That is, the freshness measurement system 1b of the second embodiment includes the measurement container 700 and the phosphor unit 301 in place of the measurement container 200 and the phosphor unit 300 of the freshness measurement system 1 of the first embodiment.

The phosphor unit 301 includes a base part 311 in place of the base part 310 of the phosphor unit 300 in the first embodiment. The base part 311 is a transparent plate-like resin, glass, or the like.

The measurement container 700 is, for example, a container for storing a food 230 such as a box for shipping marine products or a refrigerator. Alternatively, the measurement container 700 is a container for putting the food 230 therein for measuring the freshness. In one example, the measurement container 700 includes a lid 710 and a container 720.

The lid 710 includes an installation part 711. Further, the lid 710 has a hole 712.

The installation part 711 is configured to be able to detachably install the phosphor unit 301. The phosphor unit 301 installed at the installation part 711 is configured such that a fluorescent structure 320 is exposed to a gas G in the measurement container 700 through the hole 712. Further, the installation part 711 is configured such that the base part 311 closes the hole 712 so as to prevent the gas G from leaking.

An operation of the freshness measurement system 1b of the second embodiment is the same as that of the freshness measurement system 1 of the first embodiment, and therefore, a description thereof will be omitted. Note that the measurement of the freshness of the food 230 put in the measurement container 700 is performed after the elapse of a sufficient time from when the food is put in the measurement container 700. This time is, for example, from 1 hour to 2 hours.

From the measurement container 700 of the freshness measurement system 1b of the second embodiment, the phosphor unit 301 can be detached. Therefore, by replacing the phosphor unit 301, the same measurement container 700 can be repeatedly used.

The above-mentioned embodiment can also be modified as follows.

The excitation light source 120 and the light sensor 130 may be present in the measurement container 700 as shown in FIG. 17. FIG. 17 is a view showing a modification example of an arrangement of the excitation light source 120 and the light sensor 130.

The excitation light source 120 may be an annular light source with the light sensor 130 as the center. By using the annular light source, the luminance of the excitation light can be increased. Further, when using the annular light source, a shaded portion hardly occurs, and the excitation light is likely to be uniformly applied to the phosphor unit 301.

The base part 310 or the base part 311 may be a porous material. When the base part 310 or the base part 311 is a porous material, the fluorescent structure 320 may be located on the opposite side of the food across the base part 310 or the base part 311. Further, when the fluorescent structure 320 is located on the opposite side of the food across the base part 310 or the base part 311, the base part 310 or the base part 311 need not be transparent. This is because when the base part 310 or the base part 311 is a porous material, the fluorescent structure 320 can come into contact with a subject component through the pores of the base part 310 or the base part 311. The material of the base part 310 or the base part 311 in such a case may be, for example, a plastic sheet, paper, cloth, sponge, or the like. Further, the material of the base part 310 or the base part 311 may be the same material as the material of the base material 321.

In the above-mentioned embodiment, a part of the processing performed by the processing device 400 may be performed by the measurement device 100. Further, in the above-mentioned embodiment, apart of the processing performed by the measurement device 100 may be performed by the processing device 400.

The measurement device of the embodiment may be a device that serves as both the measurement device 100 and the processing device 400 in the above-mentioned embodiment.

Further, the measurement device of the embodiment may be a device that serves as the measurement device 100, the processing device 400, the display device 500, and the input device 600 in the above-mentioned embodiment.

Therefore, a case where the freshness measurement system of the embodiment is a single measurement device is included. Further, a case where also the system within the scope of the claims is, similarly, a single device is included.

The freshness measurement system 1 may make a notification of the contents displayed by the display device in the above-mentioned embodiment through a method other than an image. For example, the freshness measurement system 1 may make a notification of the contents through a voice.

In the above-mentioned embodiment, the fluorescent structure 320 changes the fluorescence intensity by reacting with an organic acid and an amine compound or the like. However, the fluorescent structure 320 may be configured to change the fluorescence intensity by reacting with a substance other than an organic acid and an amine compound.

The white standard plate 330 and the black standard plate 340 may be attached to the measurement device 100 instead of the phosphor unit 300. In that case, the measurement device 100 includes the white standard plate 330 and the black standard plate 340 at positions where a light emitted by the excitation light source 120 is applied, and a reflected light can be measured by the light sensor 130.

The freshness measurement system of the embodiment need not include the white standard plate 330 and the black standard plate 340. In that case, the measurement device 100 and the processing device 400, for example, measure the fluorescence intensity as an absolute amount. That is, for example, the processor 410 uses, as the fluorescence intensity, a phosphor emission intensity Sp instead of the fluorescence intensity ratio Ip and skips the processing of ACT 19.

The freshness measurement system 1 may use a value other than the K value as the freshness index. The freshness measurement system 1 may use a fluorescence intensity as the freshness index.

The processor 410 may be configured to realize a part or all of the processing realized by the program in the above-mentioned embodiment by a hardware configuration of a circuit.

The processing device 400 in the above-mentioned embodiment is, for example, transferred to a manager or the like of each device in a state where a program for executing each processing described above is stored. Alternatively, the processing device 400 is transferred to the manager or the like in a state where the program is not stored. Then, the program is separately transferred to the manager or the like, and is stored in the processing device 400 based on an operation by the manager or a service person or the like. The transfer of the program at that time can be realized, for example, using a removable memory medium such as a disk medium or a semiconductor memory, or by downloading via the Internet, LAN, or the like.

While certain embodiments have been described, these embodiments have been presented by way of example only and are not intended to limit the scope of the invention. The novel embodiments described herein may be embodied in a variety of other forms, furthermore, various omissions, substitutions, and changes may be made without departing from the gist of the invention. These embodiments and modifications thereof are included in the scope or gist of the invention, and are included in the invention described in the claims and the equivalents thereof.

What is claimed is:

1. A freshness measurement system, comprising:
an irradiation component configured to irradiate a phosphor that changes an intensity of fluorescence according to a concentration of a component released from a test subject with an excitation light;
a measurement component configured to measure the intensity of fluorescence emitted by the phosphor; and
a processor configured to determine a freshness index of the test subject using the measured intensity.

2. The freshness measurement system according to claim 1, wherein the freshness index is a K value.

3. The freshness measurement system according to claim 1, further comprising a notification component configured to make a notification of the freshness index.

4. The freshness measurement system according to claim 1, further comprising an installation part configured to install the phosphor so that the phosphor is exposed to a gas containing the component.

5. The freshness measurement system according to claim 1, further comprising the phosphor.

6. The freshness measurement system according to claim 1, wherein the excitation light irradiated by the irradiation component comprises ultraviolet light.

7. The freshness measurement system according to claim 1, wherein the freshness index of the test subject is determined without damage or degradation of the test subject.

8. A freshness measurement method, comprising:
irradiating with an excitation light a phosphor that changes an intensity of fluorescence according to a concentration of a component released from a test subject;
measuring the intensity of fluorescence emitted by the phosphor; and
determining a freshness index of the test subject using the measured intensity.

9. The freshness measurement method according to claim 8, wherein the freshness index is a K value.

10. The freshness measurement method according to claim 8, further comprising notifying an observer of the freshness index.

11. The freshness measurement method according to claim 8, further comprising installing the phosphor so that the phosphor is exposed to a gas containing the component.

12. The freshness measurement method according to claim 8, wherein the excitation light comprises ultraviolet light.

13. The freshness measurement method according to claim 8, wherein determining the freshness index of the test subject is conducted without damage or degradation of the test subject.

14. A perishable food freshness measurement system, comprising:
an irradiation component configured to irradiate a phosphor that changes an intensity of fluorescence according to a concentration of a component released from a perishable food item with an excitation light;
a measurement component configured to measure the intensity of fluorescence emitted by the phosphor; and
a processor configured to determine a freshness index of the perishable food item using the measured intensity.

15. The perishable food freshness measurement system according to claim 14, wherein the freshness index is a K value.

16. The perishable food freshness measurement system according to claim 14, further comprising a notification component configured to make a notification of the freshness index.

17. The perishable food freshness measurement system according to claim 14, further comprising an installation part configured to install the phosphor so that the phosphor is exposed to a gas containing the component.

18. The perishable food freshness measurement system according to claim 14, further comprising the phosphor.

19. The perishable food freshness measurement system according to claim 14, wherein the excitation light irradiated by the irradiation component comprises ultraviolet light.

20. The perishable food freshness measurement system according to claim 14, wherein the freshness index of the perishable food item is determined without damage or degradation of the perishable food item.
